(19) United States
(12) Patent Application Publication
(10) Pub. No.: US 2019/0049455 A1
(43) Pub. Date: Feb. 14, 2019

(54) HAAH and MMP-9 are Complementary Cancer Biomarkers and Predictors of Metastasis when Combined

(71) Applicant: Panacea Pharmaceuticals Inc., Gaithersburg, MD (US)

(72) Inventors: Hossein Ghanbari, Gathersburg, MD (US); Mark Semenuk, Gaithersburg, MD (US)

(21) Appl. No.: 16/058,971

(22) Filed: Aug. 8, 2018

Related U.S. Application Data
(60) Provisional application No. 62/544,402, filed on Aug. 11, 2017.

(51) Int. Cl. G01N 33/574; G01N 33/573; G01N 33/543; A61K 9/127; G06F 19/24; G16H 50/30

(52) U.S. Cl. CPC G01N 33/57488; G01N 33/573; G01N 33/54326; G01N 33/5748; G06F 19/24; G16H 50/30; G01N 33/5432; A61K 9/1271

(57) ABSTRACT

The present disclosure relates to methods of using biomarkers as early disease and patient outcome predictors. More particularly, the present disclosure encompasses methods of predicting cancer metastasis by detecting and/or quantifying aspartyl (asparaginyl) beta hydroxylase (HAAH) and matrix metalloproteinase 9 (MMP9) in a biological sample.

### CROSS-REFERENCE TO RELATED APPLICATIONS

This application claims the benefit of U.S. Provisional Application No. 62/544,402, filed on Aug. 11, 2017. The content of this application is incorporated herein by reference in its entirety.

### FIELD OF THE INVENTION

The present disclosure relates to methods of using biomarkers as early disease and patient outcome predictors. More particularly, the present disclosure relates to methods of predicting cancer metastasis.

### BACKGROUND OF THE INVENTION

Cancer metastasis involves a complex series of steps in which cancer cells leave the original tumor site and migrate to other parts of the body via the bloodstream, the lymphatic system, or by direct extension. Metastasis is a very important indication of the malignancy and development stage of a tumor. However, metastatic cancer is difficult to assess because patients with metastatic cancer do not have symptoms or they have symptoms that are also common to other diseases.

Therefore, there are continuing needs to develop novel methods to detect and/or predict metastasis.

Matrix metallopeptidase 9 (MMP-9), also known as 92 kDa type IV collagenase, 92 kDa gelatinase or gelatinase B (GELB), is a matrixin, a class of enzymes that belong to the zinc-metalloproteinases family involved in the degradation of the extracellular matrix. In humans, the MMP9 gene encodes for a signal peptide, a propeptide, a catalytic domain with inserted three repeats of fibronectin type 11 domain followed by a C-terminal hemopexin-like domain.

Proteins of the matrix metalloproteinase (MMP) family are involved in the breakdown of extracellular matrix in normal physiological processes, such as embryonic development, reproduction, angiogenesis, bone development, wound healing, cell migration, learning and memory, as well as in pathological processes, such as arthritis, intracerebral hemorrhage, and metastasis. Most MMPs are secreted as inactive proproteins which are activated when cleaved by extracellular proteinases. The enzyme encoded by this gene degrades type IV and V collagens and other extracellular matrix proteins. Studies in rhesus monkeys suggest that the enzyme is involved in IL-8-induced mobilization of hematopoietic progenitor cells from bone marrow, and murine studies suggest a role in tumor-associated tissue remodeling.

Aspartyl-(Asparaginyl)-β-hydroxylase (HAAH) is over expressed in various malignant neoplasms, including hepatocellular and lung carcinomas. HAAH is a tumor specific antigen, which is specifically expressed on the surface of certain malignant cells. HAAH is a hydroxylation enzyme that modifies factors such as Notch that contribute to cancer etiology by causing cell proliferation, motility, and invasiveness. Neutralizing the enzyme or reducing its expression leads to normal phenotype(s) in cancer cells. Anti-HAAH antibodies (as well as siRNA) have been shown to be cytostatic. An all-human sequence anti-HAAH antibody (PAN-622) has shown to inhibit tumor growth by more than 90% in animal studies by passive immunotherapy. However, HAAH is well conserved and is also over expressed in placenta hence it is not sufficiently immunogenic in animals and it is certainly a self-antigen in humans.

Cancer-specific cell surface HAAH functions by enzymatically modifying a number of motif-restricted protein targets including Notch. It thereby triggers events leading to metastasis. MMP9 is a well-known enabler of metastasis due to its inherent effect on the process of proteolytically-assisted tumor cell escape, albeit not as useful as a cancer biomarker on its own. The present disclosure proposes that up-regulated HAAH is a prerequisite for metastasis and that in turn MMP9 is an enabler of this process.

### SUMMARY OF THE INVENTION

The present disclosure relates to methods of using biomarkers as early disease and patient outcome predictors.

The present invention contemplates methods of predicting cancer metastasis.

The present invention further contemplates methods for evaluating whether a subject is at risk of suffering from metastasis.

Further, the present invention provides methods of quantifying the presence of biomarkers as a way of evaluating the probability of metastasis in a subject.

The present invention further contemplates the use of complementary biomarkers associated with mediators of cancer cell mobility and invasiveness for early disease and patient outcome predictors.

The present invention encompasses methods of developing a metastatic score based on the presence of complimentary biomarkers associated with mediators of cancer cell mobility and invasiveness.

One embodiment of the present invention encompasses a method of predicting cancer metastasis in a patient comprising the steps of analyzing a biological sample from the patient to determine if the biological sample contains HAAH and MMP9.

In certain embodiments of the present invention, blood levels of HAAH combined with those of MMP9 are used to determine a metastatic score to be used in patient management.

Another embodiment of the present invention encompasses methods of detecting serum and exosomal HAAH and MMP9 through enzyme-linked immunosorbent assay (ELISA).

The present invention further provides a quantitative assessment of HAAH and MMP9 in serumlserum exosomes from cancer patients to evaluate their concerted role in metastasis and to formulate a metastatic score.

One embodiment of the present invention encompasses a method for predicting metastasis in a subject comprising the steps of obtaining a biological sample from the subject, detecting if there is HAAH in the biological sample and detecting if there is MMP9 in the biological sample, wherein the presence of HAAH and MMP9 in the biological sample indicates an increased probability of metastasis.

Another embodiment of the present invention encompasses a method for predicting the probability of metastasis in a subject comprising the steps of obtaining a biological sample from the subject, quantifying the level of HAAH in the biological sample, quantifying the level of MMP9 in the biological sample, and determining a metastatic score based on the levels of HAAH and MMP9, wherein the metastatic score indicates probability of metastasis in the subject.

An embodiment of the invention encompasses a kit for determining the probability of metastasis in a subject. The kit comprises materials that can detect in a biological sample from the subject, the presence of HAAH and the presence of MMP9, and instructions for carrying out an in-vitro determination of the presence of HAAH and MMP9 in the biological sample.

### BRIEF DESCRIPTION OF THE FIGURES

FIGS. 1A to 1C show a diagram of the HAAH assay workflow. FIG. 1A: disruption of cells and separation of exosomes; FIG. 1B: binding of exosomes to FB50 antibody conjugated to biotin and streptavidin; FIG. 1C: reaction of labeled exosomes with FB50 pre-coated microplates.

FIG. 2 shows a graph of a typical ELISA standard calibration curve using recombinant HAAH (rHAAH). The level of HAAH in ng/ml is on the X axis, and the absorbance readings at 450 nm are on the Y axis. Results obtained by two different analysts are shown (analyst 1 (□), analyst 2 (◯)).

FIG. 3 depicts a graph of a typical ELISA standard calibration curve using recombinant MMP9 (rMMP9). The amounts of MMP9 in ng/ml are on the X axis, and the absorbance readings at 450 nm are on the Y axis.

FIGS. 4A and 4B show graphs resulting from NANOSIGHT nanoparticle analysis of exosomes. FIG. 4A: analysis of exosomes prepared from a healthy donor serum; FIG. 4B: analysis of exosomes prepared from a breast cancer serum pool. The particle size in nm is on the X axis, and the particle concentration in particles/ml is on the Y axis.

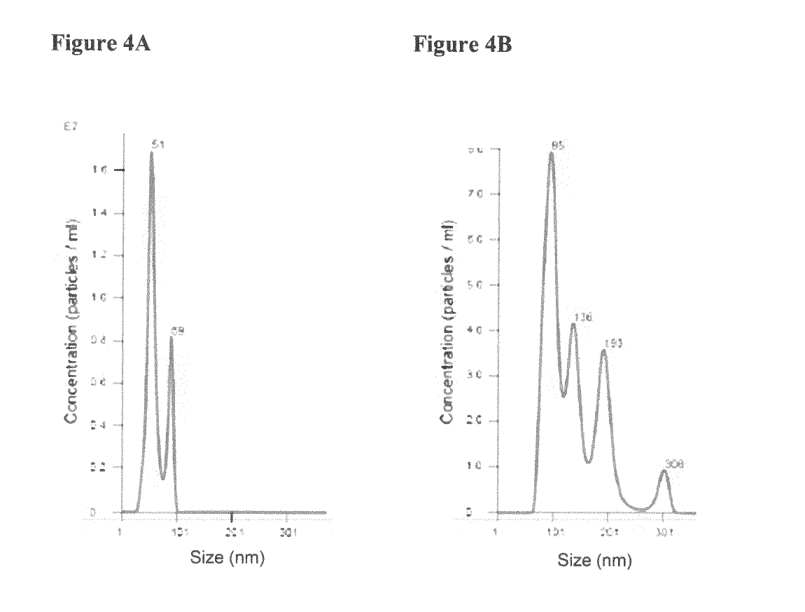

FIG. 5 shows the HAAH determinations on high-risk volunteers. The amount of HAAH in ng/ml is on the Y axis. Samples positive for both, HAAH and MMP9, are shown by dark circles (•); samples positive for HAAH but negative for MMP9 are shown by lighter circles (◯); samples negative for both, HAAH and MMP9, are shown as open circles (◯). A horizontal solid line indicates the cut-off value for HAAH (3 ng/ml). The dashed line labeled “PC” is the ELISA readout for the positive control, and the dashed line labeled “NC” is the ELISA readout for the negative control. The value 0 is indicated by a dotted line.

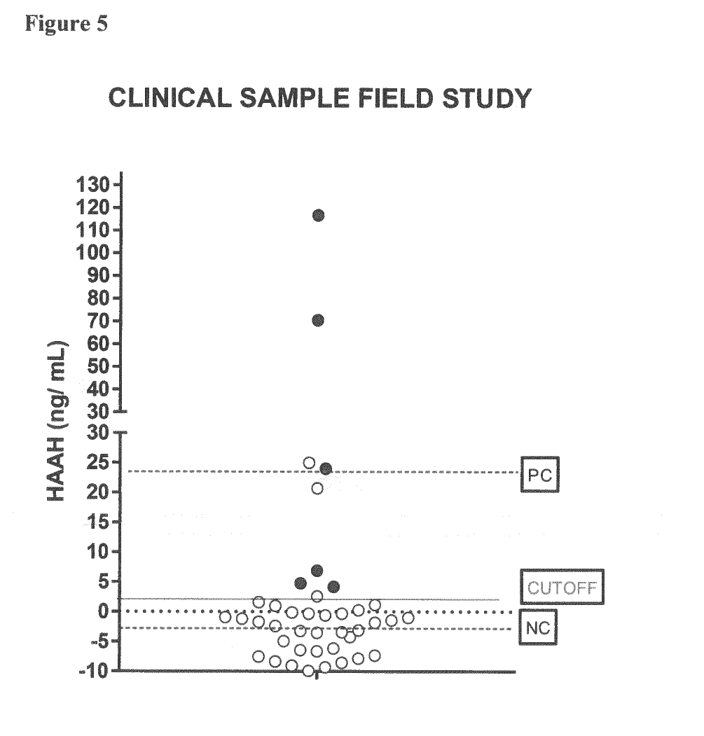

FIG. 6 depicts a plot of the relationship between HAAH and MMP9 among the mixed commercial BIORECLAMATION cancer samples. Denoted with arrows and solid circles are samples from patients with known metastatic disease, as indicated in Table 2. The numbers next to the arrows correspond to the numbers as listed in Table 3. The amount of MMP9 in ng/ml is on the X axis, and the amount of HAAH in ng/ml is on the Y axis. A horizontal dashed line indicates the cut-off value for HAAH (3 ng/ml); and a vertical dashed line indicates the cut-off value for MMP9 (100 ng/ml).

FIG. 7 depicts a plot of the relationship between HAAH and MMP9 among the samples from cancer high-risk volunteers in an ongoing field study. Samples were obtained from the volunteers twice, six (6) weeks apart. Denoted with a C and a D are the HAAH and MMP9 relationships in samples from volunteer H44 taken 6 weeks apart. Levels of MMP9 in ng/ml are on the X axis, and levels of HAAH in ng/ml are on the Y axis. A horizontal dashed line indicates the cut-off value for HAAH (3 ng/ml); and a vertical dashed line indicates the cut-off value for MMP9 (100 ng/ml). Samples positive for both, HAAH and MMP9, appear above and to the right of the dashed lines.

FIGS. 8A and 8B depict graphs of the NANOSIGHT nanoparticle analyses results of exosomes from field study volunteer H44. FIG. 8A: graph of results before resolution of HAAH and MMP9 biomarker levels. FIG. 8B: graph of results after resolution of HAAH and MMP9 biomarker levels. Particle size in nm is on the X axis, and concentration in particles/ml is on the Y axis.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the methods and materials used are now described. All publications mentioned herein are incorporated herein by reference in their entirety.

For avoidance of doubt, the term “metastasis” indicates the development of additional tumor growths at a distance from a primary site of cancer.

In the present disclosure, “MMP-9” and “MMP9” are used interchangeably and refer to the matrix metallopeptidase 9, also known as 92 kDa type IV collagenase, 92 kDa gelatinase or gelatinase B (GELB). Matrix metallopeptidase 9 is a matrixin, a class of enzymes that belong to the zinc-metalloproteinases family involved in the degradation of the extracellular matrix. In humans the MMP9 gene encodes for a signal peptide, a propeptide, a catalytic domain with inserted three repeats of fibronectin type II domain followed by a C-terminal hemopexin-like domain.

The Malvern Panalytical (Malvern, United Kingdom) NANOSIGHT instruments are said to provide an easy-to-use, reproducible platform for nanoparticle characterization.

Exosomes may be defined as extracellular vesicles that are released from cells upon fusion of an intermediate endocytic compartment, the multivesicular body, with the plasma membrane. This liberates intraluminal vesicles (ILVs) into the extracellular milieu and the vesicles thereby released are what is currently known as exosomes. Methods for isolating exosomes are known in the art and are taught, for example, in U.S. Pat. No. 8,901,284; U.S. Pat. No. 9,005,888; and Théry C. et al. (2006, “Isolation and Characterization of Exosomes from Cell Culture Supernatants and Biological Fluid,” Curr. Protoc. Cell Biol. 3 (2): 22).

The present invention provides methods for evaluating or predicting the likelihood that a subject having cancer will experience metastasis. The present disclosure is based on the discovery that the presence of certain complementary biomarkers can be used to assess the risk of metastasis in a subject. Together, complementary biomarkers associated with mediators of cancer cell mobility and invasiveness can be used as early disease and patient outcome predictors. The present disclosure provides a quantitative assessment of HAAH [aspartyl (asparaginyl) beta hydroxylase] and MMP9 [matrix metalloproteinase 9] in serum/serum exosomes from cancer patients to evaluate their concerted role in metastasis and formulate a metastatic score.

In some embodiments, the subject is a mammal. A mammal may be a domesticated animal (e.g., cow, sheep, cat, dog, or horse), a primate (e.g., a human or a non-human primate such as a monkey), a rabbit, or a rodent (e.g., a mouse or a rat). In some embodiments, the mammal is a human.

In some embodiments, the subject suffers from a cancer selected from the group consisting of breast cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. Thus, the method for the present invention is generally applicable to any type of cancer in which epithelial-mesenchymal transition (EMT) occurs.

In some embodiments, the biological sample is a fluid sample from the subject. The biological sample may be any fluid such as blood, saliva, urine, pleural effusion, semen, or breast discharge. In some embodiments, the biological sample is a blood sample. By “blood sample” is meant a volume of whole blood or fraction thereof, eg, serum, plasma, etc. In some embodiments the biological sample is serum.

The ectopic expression of human Aspartyl (Asparaginyl) ß-Hydroxylase (HAAH) as a serum cancer biomarker closely parallels significant cancer cell specific events such as cellular differentiation, motility, and metastasis (Ince N., et al., 2000, “Overexpression of human aspartyl (asparaginyl) ß-hydroxylase is associated with malignant transformation,” Cancer Res. 60:

The biomarkers discussed herein may be detected and/or quantified by any method known presently in the art. Exemplary methods include, but are not limited to spectrometry methods, high-performance liquid chromatography (HPLC), liquid chromatography-mass spectrometry (LC/MS), antibody dependent methods, enzyme-linked immunosorbent assay (ELISA), protein immunoprecipitation, immunoelectrophoresis, protein immunostaining.

The present invention encompasses methods for predicting metastasis in a subject comprising the steps of:

obtaining a biological sample from the subject,

detecting if there is HAAH in the biological sample and

detecting if there is MMP9 in the biological sample,

wherein the presence of both, HAAH and MMP9, in the biological sample indicates an increased probability of metastasis.

The present invention also encompasses methods for predicting the probability of metastasis in a subject comprising the steps of:

obtaining a biological sample from the subject,

quantifying the level of HAAH in the biological sample,

quantifying the level of MMP9 in the biological sample, and

determining a metastatic score based on the levels of HAAH and MMP9, wherein the metastatic score indicates probability of metastasis in the subject.

In some embodiments, thee metastatic score is calculated as:

Metastatic Score=[HAAH (ng/ml)×10+MMP9 (ng/ml)]100

The present invention encompasses a kit for determining the probability of metastasis in a subject. The kit comprises materials that can detect the presence of HAAH and materials that can detect the presence of MMP9 in a biological sample from the subject, and instructions for carrying out an in-vitro determination of the presence of HAAH and MMP9 in the biological sample.

An embodiment of the invention encompasses a kit for determining the probability of metastasis in a subject. The kit comprises materials that can detect the presence of HAAH and materials that can detect the presence of MMP9 in a biological sample from the subject, and instructions for carrying out an in-vitro determination of the presence of HAAH and MMP9 in the biological sample.

In an embodiment, the invention provides kits for determining the probability of tumor cells in a subject undergoing metastasis, where the kit includes an agent that specifically binds to HAAH and an agent that specifically binds to MMP9. In some embodiments, the agents that bind HAAH and MMP9 are monoclonal antibodies. The kit may also include instructions for using the agent that specifically binds to HAAH and the agent that specifically binds to MMP9 to determine in vitro the presence of HAAH and MMP9 in the subject.

In some embodiments the kit uses antibodies as capture reagents. In some embodiments, a substrate (e.g., a multiwell plate) can have a specific HAAH and/or MMP9 capture reagent attached thereto. In some embodiments, a kit can have a blocking reagent included. Blocking reagents can be used to reduce non-specific binding. For example, non-specific antibody binding can be reduced using an excess of a blocking protein such as serum albumin. It can be appreciated that numerous methods for detecting peptides and proteins are known in the art, and any strategy that can specifically detect HAAH and MMP9 molecules can be used and be considered within the scope of this invention.

Methods for the quantitation of proteins are known in the art. For example, antibodies may be used to determine HAAH and MMP9 quantitation using western blots, immunohistochemistry, immunocytochemistry, flow cytometry, immunoprecipitation, immunoassay, functional assay, Enzyme Linked Immunosorbent Assay (ELISA), Electrophoretic Mobility Shift Assay (EMSA), among others.

In some embodiments, HAAH and MMP9 may be detected in a biological sample by adding magnetic beads coated with an HAAH specific antibody and magnetic beads coated with an MMP9 specific antibody to the biological sample, and analyzing the magnetic beads for the presence of HAAH and MMP9.

In some embodiments, a kit for determining the probability of tumor cells undergoing metastasis comprises magnetic beads coated with HAAH and magnetic beads coated with MMP9. In some embodiments, the kit includes instructions for determining the presence of HAAH and of MMP9. In some embodiments the kit includes instructions for calculating a risk score that the cells will undergo metastasis.

There is long-standing interest in elevated serum MMP9 as a biomarker that may have predictive value in assessing metastatic progression in a number of cancers (Vihinen P. and Kahari V. M., 2002, “Matrix metalloproteinases in cancer: prognostic markers and therapeutic targets,” Int. J. Cancer 99:157). However this elevation is sometimes non-specific, as a heightened expression can occur in destructive inflammatory tissue diseases other than cancer, such as arthritis (Gruber B. L., et al., 1996, “Markedly elevated serum MMP-9 (gelatinase B) levels in rheumatoid arthritis: a potentially useful laboratory marker,” Clin. Immunol. Immunopathol. 78: 161), and vasculitis (Takeshita S., et al., 2001, “Elevated serum levels of matrix metalloproteinase-9 (MMP-9) in Kawasaki disease,” Clin. Exp. Immunol. 125: 340). The present investigation therefore seeks out to combine MMP9 and a complementary biomarker, HAAH, which has been shown to have a good general predictive value for cancer.

Despite expectations that both MMP9 and HAAH should be similar, and could be recovered in the serum exosomal compartment, their relative expression was not always directly correlated in serum samples. This allows a unique ability to stratify HAAH scoring as being associated or not with possible metastatic disease.

### EXAMPLE

### Methods

We detect serum and exosomal HAAH by a simultaneous-homologous ELISA format using an in house manufactured reagent kit comprising pre-coated microplates and pre-formulated reagents. Serum and exosomal MMP9 was detected with a commercial reagent kit ELISA (Abeam; Cambridge, United Kingdom). Exosomes were prepared using a 50% polyethylene glycol 6000/0.5 M NaCl solution added to serum, centrifugation, and reconstitution. CEA positive cancer and healthy serum samples were obtained commercially (Complex Antibodies; Margate, U.S.A) or through off site collaborators.

### Preparation of Exosomes

Exosomes were prepared from serum by a method essentially as described by Manri et al (2017, “Size-Selective Harvesting of Extracellular Vesicles for Strategic Analyses Towards Tumor Diagnoses,” Appl. Biochem. Biotechnol. 182: 609) using a 10% net final concentration of Polyethylene Glycol 6000. Fifty microliters (50 μl) (or multiples of this volume) from each serum sample or control was mixed with 10 μL (or multiples thereof) of 50% polyethylene glycol 6000 in 0.5 M NaCl. After a 10 minute incubation at room temperature, the samples were centrifuged at 10,000×g for 10 minutes. After aspirating the supernatant, the exosomal pellets were reconstituted with either 50 μL Phosphate Buffered Saline (PBS) or 50 μL pooled normal serum (Innovative Research Inc.; Novi, Mich., U.S.A.). Exosomes prepared in this manner were evaluated using a NANOSIGHT nanoparticle tracking analysis instrument (Malvern Panalytical, Malvern, United Kingdom).

### HAAH ELISA

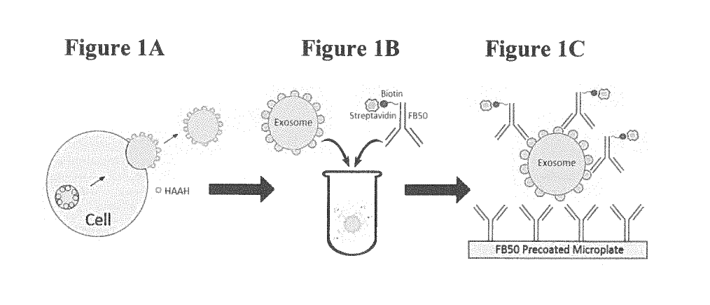

The HAAH ELISA was carried out using pre-formulated buffers, reagents, and Mylar-packaged pre-coated microplates in a reagent kit format. A workflow diagram of the HAAH assay is depicted in FIGS. 1A to 1C. The assay uses the same anti-HAAH antibody (FB50) for capture and detection steps in a homologous microplate format. The FB50 antibody was initially raised against the hepatoma cell line FOCUS and has been described previously (Lavaissiere, L., et al., 1996, “Overexpression of Human Aspartyl (Asparaginyl)b-Hydroxylase in Hepatocellular Carcinoma and Chomangiocarcinoma,” J. Clin. Invest. 98: 1313).

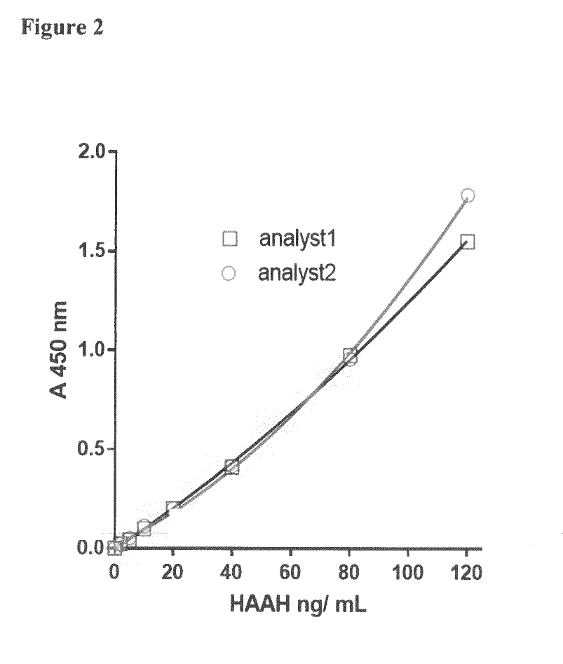

The FB50 antibody was produced using the hybridoma cell line having American Type Culture Collection (ATCC) accession number PTA 3386. Recombinant HAAH (rHAAH) was prepared as an affinity-purified baculovirus-expressed protein, and served as assay calibrator. In the ELISA assay exosomes, prepared as above, were incubated in the presence of FB50 antibody labeled with biotin and streptavidin, reacted with an FB50-coated microplate, and visualized. A graph of a typical ELISA rHAAH standard calibration curve is depicted in FIG. 2. The results obtained by two different analysts are shown.

### MMP-9 ELISA

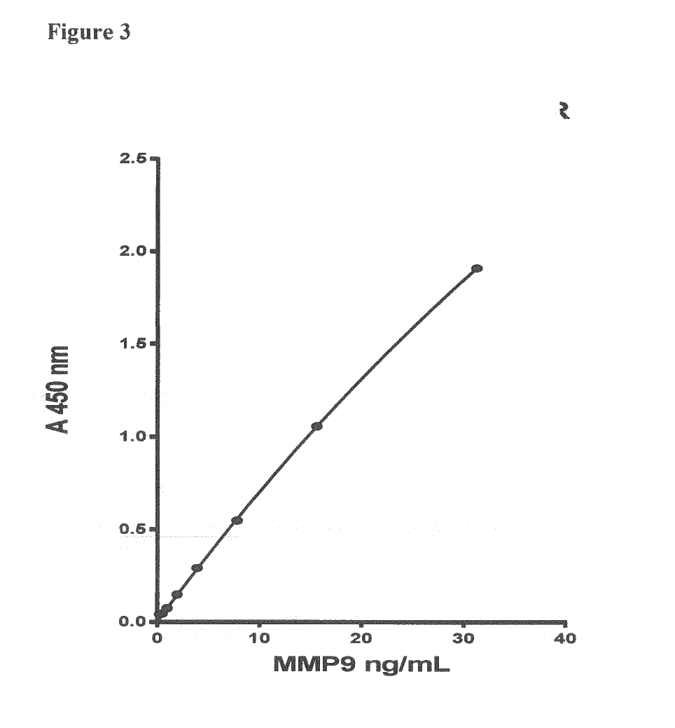

An MMP9 ELISA reagent kit (Abcam) comprising capture antibodies, detection antibodies, and all the raw materials was utilized for the serum and exosome MMP9 quantification. A graph of a typical rMMP9 standard calibration curve is shown in FIG. 3.

Samples tested were either frozen archived serum or fresh serum received from an off-site clinical laboratory. The off-site samples were shipped via overnight courier to the laboratory prior to testing in the field study.

Table 2, below, lists the characteristics of the samples in a BIORECLAMATION commercial cancer serum set (BIORECLAMATION, Hicksville, N.Y., U.S.A.), the measured HAAH and MMP9 levels, and the calculated metastatic risk score in these samples. This BIORECLAMATION commercial cancer serum set is derived from a mixed selection of cancers (lung, prostate, breast).

TABLE 2
BIORECLAMATION CANCER SERUM SET CHARACTERISTICS,
HAAH AND MMP9 LEVELS, METASTATIC SCORE

| Lot # | HAAH | MMP9 | Age | Stage | Type | Metastatic | Score |
|---|---|---|---|---|---|---|---|
| BRH653948 | 60.5 | 599.4 | 82 | 4 | Lung | Yes, lymph | 1204.4 |
| BRH653949 | 29.9 | 206.6 | N/A | 4 | Lung | Yes, bone | 505.6 |
| BRH653950 | * | 83.8 | 81 | 4 | Lung | Yes, bone | * |
| BRH653952 | 8.3 | 83.8 | 79 | 4 | Lung | N/A | 166.8 |
| BRH653953 | 6.7 | 54.9 | 72 | 2 | Lung | N/A | 121.9 |
| BRH653954 | 10.6 | 53.3 | 84 | 4 | Lung | N/A | 159.3 |
| BRH653955 | 14.4 | 56.5 | 73 | 4 | Lung | N/A | 200.5 |
| BRH653956 | 2 | 33.3 | 64 | 3 | Lung | N/A | 53.3 |
| BRH653957 | 36.7 | 30.9 | 65 | 3 | Lung | N/A | 397.9 |
| BRH653959 | 2 | 112.9 | 70 | 4 | Lung | N/A | 132.9 |
| BRH653960 | 20.9 | 34.1 | 69 | 2 | Prostate | N/A | 243.1 |
| BRH653961 | 3.4 | 108.1 | 78 | 1 | Prostate | N/A | 142.1 |
| BRH653964 | 32.4 | 90.3 | 63 | T1C | Prostate | N/A | 414.3 |
| BRH653965 | 125 | 70.9 | 87 | N/A | Prostate | N/A | 1320.9 |
| BRH653966 | 72.1 | 46.13 | 86 | N/A | Prostate | N/A | 767.13 |
| BRH653967 | 2 | 200 | 78 | 4 | Prostate | N/A | 220 |
| BRH653969 | * | 142.3 | 69 | N/A | Prostate | N/A | * |
| BRH653970 | 26.2 | 58 | 66 | 4 | Prostate | N/A | 320 |
| BRH653971 | 12.1 | 121.9 | 75 | 4 | Prostate | Yes | 242.9 |
| BRH653974 | * | 182.6 | 57 | T1C | Prostate | No | * |
| BRH653975 | 9.6 | 71.7 | 74 | N/A | Prostate | No | 167.7 |
| BRH653979 | 13.1 | 411.1 | 68 | T2B | Prostate | unk | 542.1 |
| BRH653980 | 45.3 | 118.6 | 78 | N/A | Prostate | N/A | 571.6 |
| BRH653981 | 71.6 | 158.7 | 63 | T3A | Prostate | N/A | 874.7 |
| BRH653982 | 15.6 | 111.3 | 84 | 4 | Prostate | N/A | 267.3 |
| BRH653984 | 46.2 | 61.3 | 75 | N/A | Prostate | N/A | 523.3 |
| BRH653986 | 72.1 | 46.1 | 48 | T1C | Prostate | N/A | 767.1 |
| BRH653989 | 6.4 | 102.4 | 73 | 4 | Prostate | N/A | 166.4 |
| BRH653991 | 54 | 166.1 | 71 | N/A | Prostate | N/A | 706.1 |
| BRH653992 | 16.2 | 343.5 | 86 | 4 | Prostate | N/A | 505.5 |
| BRH653993 | 17.1 | 143.9 | 63 | N/A | Prostate | N/A | 314.9 |
| BRH653995 | 2 | 168.6 | 68 | T1C | Prostate | N/A | 188.6 |
| BRH653948 | 60.5 | 599.4 | 82 | 4 | Lung | Yes, lymph | 1204.4 |
| BRH653949 | 29.9 | 206.6 | N/A | 4 | Lung | Yes, bone | 505.6 |
| BRH653996 | 9.8 | 70.1 | 72 | 4 | Prostate | N/A | 168.1 |
| BRH653997 | 7.6 | 41.3 | 82 | 1 | Prostate | N/A | 117.3 |
| BRH653998 | 21.1 | 58.9 | 67 | N/A | Prostate | N/A | 269.9 |
| BRH654000 | 61.5 | 161.2 | 64 | N/A | Prostate | N/A | 776.2 |
| BRH654003 | 4.1 | 97.5 | 81 | N/A | Prostate | N/A | 138.5 |
| BRH654004 | 31.6 | 234.1 | 70 | N/A | Prostate | N/A | 550.1 |
| BRH654005 | 2 | 113.8 | 78 | N/A | Prostate | N/A | 133.8 |
| BRH654007 | 2 | 228.3 | 79 | 1 | Prostate | N/A | 248.3 |
| BRH654008 | 16.7 | 359.9 | 72 | T1C | Prostate | N/A | 526.9 |
| BRH654020 | 16.3 | 180.9 | 63 | 2 | Breast | N/A | 343.9 |
| BRH654021 | 6.4 | 95.1 | 66 | 4 | Breast | No | 159.1 |
| BRH654022 | 2 | 240.9 | 72 | 1 | Breast | No | 260.9 |
| BRH654028 | 9.6 | 131.7 | 49 | N/A | Breast | N/A | 227.7 |
| BRH654029 | 8.4 | 340.9 | 63 | 1 | Breast | N/A | 424.9 |
| BRH654030 | 2 | 187.5 | 67 | 3 | Breast | Yes, lymph | 207.5 |
| BRH654032 | * | 80.6 | 68 | 1 | Breast | unk | * |
| BRH654033 | 8.2 | 240 | 72 | N/A | Breast | N/A | 322 |
| BRH654034 | 38.4 | 283.8 | 74 | 0 | Breast | No | 667.8 |
| BRH654037 | 4.7 | 82.2 | 65 | N/A | Breast | Yes, adrenal | 129.2 |
| BRH654041 | 12.7 | 123.5 | 56 | 4 | Breast | Yes, axillary | 250.2 |
| BRH654042 | 8.4 | 42.9 | 69 | 2 | BReast | unk | 126.9 |
| BRH654044 | 2 | 74.9 | 47 | 2 | Breast | No | 94.9 |
| BRH654046 | 96.4 | 247.6 | 54 | N/A | Breast | unk | 1211.6 |
| BRH654048 | 9.1 | 118.6 | 46 | 2 | Breast | No | 209.6 |
| BRH654049 | 16 | 128.4 | 32 | 3 | Breast | Yes, lymph | 288.4 |
| BRH654051 | 34.2 | 80.6 | 70 | N/A | Breast | unk | 422.6 |
| BRH654052 | 52.7 | 148.8 | 79 | T1N1Mo | Breast | unk | 675.8 |

* Not determined

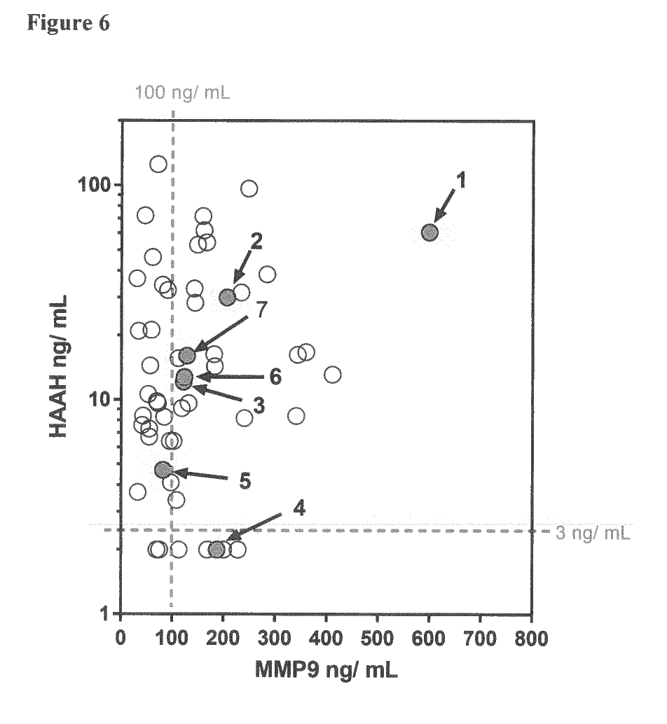

A plotted relationship of the HAAH and MMP9 levels presented in Table 2 is shown in FIG. 6. There did not appear to be a direct correction between the levels of MMP9 and HAAH, with most of the samples falling below the cut-off levels for HAAH and MMP9 (vertical and horizontal dashed lines). Denoted with arrows and solid circles are the samples from patients with known metastatic disease as outlined above in Table 2. The data for these samples is also in Table 3, below, where the type of metastasis is also listed.

Albeit limited, the known information about metastatic disease in the BIORECLAMATION set indicates that 5 out of every 7 samples (71%) can be scored according to the cutoffs given as both HAAH and MMP9 positive. Moreover, only 1 out of 19 samples (5%) that were positive for HAAH and negative for MMP9 had known metastatic disease.

A subset of the data from Table 2 is presented below in Table 3. This table lists only the samples from the BIORECLAMATION set which are known to be positive for metastasis. This table also depicts the metastatic score and the risk of metastasis in each of the samples. The metastatic score was calculated using the HAAH and MMP9 levels obtained with a NANOSIGHT instrument. The metastatic score was calculated using the formula:

Metastatic Score=[HAAH (ng/ml)×10+MMP9 (ng/ml)]/100

A metastatic score less than 2 was given a risk value of 1; a metastatic score of at least 2 but less than 3 was given a risk value of 2; a metastatic score of at least 3 but less than 4 was given a risk value of 3; a metastatic score of at least 4, but less than 5 was given a risk value of 4; and a metastatic score of 5 and above was given a risk value of 5.

TABLE 3
HAAH and MMP9 in Metastatic Samples

| | | | | | HAAH | MMP9 | | |
|---|---|---|---|---|---|---|---|---|
| | Sample | Age | Cancer | Metastasis | (ng/ml) | (ng/ml) | Score | Risk |
| 1 | BRH653948 | 82 | lung | lymph node | 60.5 | 599.4 | 1204.4 | 5 |
| 2 | BRH653949 | * | lung | bone | 29.9 | 206.6 | 505.6 | 5 |
| 3 | BRH653971 | 75 | prostate | Lung, bone | 12.1 | 121.9 | 243 | 2 |
| 4 | BRH654030 | 67 | breast | lymph node | 2.0 | 187.5 | 207 | 2 |
| 5 | BRH654037 | 65 | breast | adrenal gland | 4.7 | 82.2 | 130 | 1 |
| 6 | BRH65041 | 56 | breast | lymph node | 12.7 | 123.5 | 250 | 2 |
| 7 | BRH654049 | 32 | breast | lymph node | 16.0 | 128.4 | 290 | 2 |

* Not available
N/A Not applicable

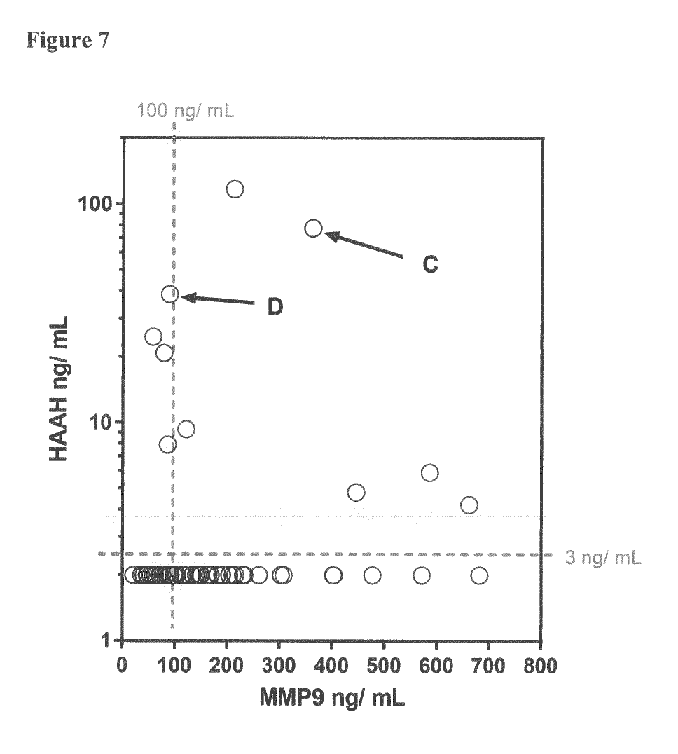

The data for the HAAH and MMP9 levels obtained in samples from an ongoing study of 48 high-risk volunteers is shown in FIG. 7. While most of the samples showed HAAH and MMP9 expression below the cut-off level, nine of the samples were found to be HAAH positive (circles above the 3 ng/ml HAAH cut-off line), and six of the samples were found to be both HAAH and MMP9 positive (circles above the 3 ng/ml HAAH cut-off line and to the right of the 100 ng/ml MMP9 cut-off line). One of the high-risk volunteers (H44) presented with high HAAH (77.4 ng/ml) and high MMP9 (363.0 ng/ml), which changed over a period of 6 weeks to 38.5 ng/ml HAAH and 89.4 ng/ml MMP9 as indicated by C and D in FIG. 7.

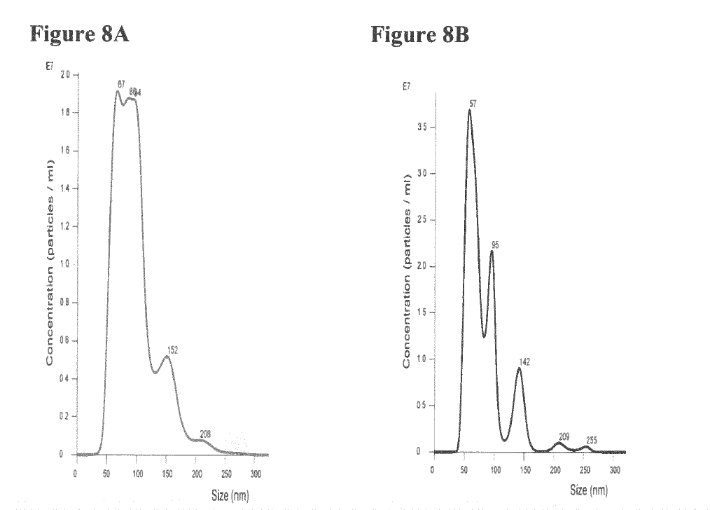

The changes seen between the samples from high risk volunteer H44 taken 6 weeks apart were associated with a more normalized exosome pattern obtained using NANOSIGHT exosome sizing instrument (Salisbury, United Kingdom). FIG. 8A shows the concentration distribution of nanoparticles in the exosomes from field study volunteer H44 before resolution of the HAAH and MMP9 levels. FIG. 8B shows the concentration distribution of nanoparticles in the exosomes from the same field volunteer after resolution of the HAAH and MMP9 levels using NANOSIGHT sizing.

### CONCLUSIONS

HAAH and MMP9 are both expected to be closely associated with metastatic activity of cancer cells, both co-localize in cancer derived exosomes, and both appear to be regulated by the same transcription factor(s). The expression of HAAH and MMP9 in serum samples is mostly coincident but sometimes may differ. This may explain differences in metastatic potential. These studies are focused upon determining whether using both biomarkers could lead to a more accurate prediction of metastatic potential. Blood levels of HAAH combined with those of MMP9 can provide a metastatic score to be used in patient management.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method for determining the probability of metastasis in a subject comprising:
obtaining a biological sample from the subject,
detecting the presence of Aspartyl-(Asparaginyl)-β-hydroxylase (HAAH) in the biological sample,
detecting the presence of Matrix metallopeptidase 9 (MMP-9) in the biological sample,
wherein the presence of both, HAAH and MMP9, in the biological sample indicates an increased probability of metastasis.

2. A method for calculating a metastasis score in a subject comprising:
detecting HAAH and MMP9 levels in a biological sample from the subject,
if the detected HAAH and MMP9 levels in the biological sample are measurable,
multiplying the subject's HAAH level by 10 to obtain a normalized HAAH level,
adding to the normalized HAAH level the subject's MMP9 level to obtain a total biomarker level,
dividing the total biomarker level by 100, and rounding down to obtain a metastatic score,
wherein the risk of metastasis increases as the value of the metastatic score increases.

3. The method of claim 1, wherein the subject is a mammal.

4. The method of claim 3, wherein the subject is a human.

5. The method of claim 1, wherein the biological sample is a bodily fluid.

6. The method of claim 5, wherein the bodily fluid is selected from the group consisting of blood, blood fraction, saliva, urine, pleural effusion, semen, or breast discharge.

7. The method of claim 6, wherein the bodily fluid is a blood fraction selected from serum and plasma.

8. The method of claim 7, wherein the bodily fluid is serum.

9. The method of claim 1, wherein exosomes are prepared from the biological sample.

10. The method of claim 2, wherein the metastatic score is 5 or higher.

11. The method of claim 1, wherein the levels of HAAH and MMP9 are detected using antibodies to HAAH and MMP9.

12. The method of claim 11, wherein HAAH and MMP9 are detected using an ELISA assay.

13. A kit for determining the probability of metastasis in a subject, the kit comprising reagents for the detection and quantification of HAAH, reagents for the detection and quantification of MMP9, and instructions for the in vitro detection and quantification of HAAH and MMP9.

14. The kit of claim 13, wherein the kit comprises magnetic beads coated with an anti-HAAH-specific antibody, and magnetic beads coated with an MMP9-specific antibody.
